(19) United States
(12) Patent Application Publication
(10) Pub. No.: US 2023/0292977 A1
(43) Pub. Date: Sep. 21, 2023

(54) ANTIVIRAL AND/OR ANTIBACTERIAL ABRASIVE BLANKET, ANTIVIRAL AND/OR ANTIBACTERIAL CLEANING SPONGE, METHOD FOR MANUFACTURING AN ANTIVIRAL AND/OR ANTIBACTERIAL ABRASIVE BLANKET AND FOR MANUFACTURING AN ANTIVIRAL AND/OR ANTIBACTERIAL CLEANING SPONGE, AND USE OF AN ANTIVIRAL AND/OR ANTIBACTERIAL ABRASIVE BLANKET AND OF AN ANTIVIRAL AND/OR ANTIBACTERIAL CLEANING SPONGE

(71) Applicant: British Indústria e Comércio Ltda, Indaiatuba (BR)

(72) Inventor: Francisco Eduardo Fonseca GORNI, Indaiatuba (BR)

(73) Assignee: British Indústria e Comércio Ltda, Indaiatuba (BR)

(21) Appl. No.: 18/009,004

(22) PCT Filed: Jun. 9, 2021
(86) PCT No.: PCT/BR2021/050250

§ 371 Date: Dec. 8, 2022

(30) Foreign Application Priority Data
Jun. 9, 2020 (BR) 10 2020 011534 0

(51) Int. Cl. A47L 13/16; B82Y 30/00

(52) U.S. Cl. CPC A47L 13/16; B82Y 30/00

(57) ABSTRACT

The present invention relates to an antiviral and/or antibacterial abrasive blanket and an antiviral and/or antibacterial cleaning sponge, in addition to their respective uses. Additionally, the present invention also relates to processes for manufacturing an abrasive blanket with an antiviral and/or antibacterial agent and for manufacturing a cleaning sponge with an antiviral and/or antibacterial agent.

The present invention relates to an antiviral abrasive blanket and/or antibacterial and an antiviral and/or antibacterial cleansing sponge, and their respective uses.

Additionally, the present invention also relates to processes for making an antiviral and/or antibacterial abrasive blanket and for making an antiviral and/or antibacterial cleaning sponge.

### FUNDAMENTALS OF THE INVENTION

In the domestic cleaning sector, different compositions and chemical formulations are used together with utensils, such as abrasive blankets and sponges, and/or with equipment, such as dishwashers and vacuum cleaners, in order to help clean and maintain surfaces and fabrics.

In this sense, the volume of abrasive/non-abrasive and cleaning sponges sold per year have been growing steadily due to their effective and practical cleaning of surfaces in general.

It is often seen on the market that cleaning sponges comprise a double-sided structure, one side being soft for a more delicate cleaning and the other side endowed with mineral properties, so that the effective cleaning of surfaces can be carried out. This tool can be found in the most varied formats, colors, softness, in addition to being able to contain an abrasive or non-abrasive layer.

Document BR 20 2014 009872-6, for example, refers to a dishwashing sponge made up of a rectangular body without abrasion and glued on its lateral faces other sponges of different abrasiveness: green (more abrasive) and blue (less abrasive).

Document BR 13 2015 017009-1 addresses developments applied in the process of obtaining a cleaning sponge with at least two layers of abrasive blankets, identical or different from each other, juxtaposed on a base; said layers being made in such a way as to compose longitudinal or transverse tufts, bringing durability, as well as providing quality in cleaning surfaces in general.

However, the surface of such utensils is subject to growth and proliferation of different microorganisms. Therefore, it is necessary to add antimicrobial agents to these materials.

It is widely known that antimicrobial agents of broad spectrum have several benefits when incorporated into materials and surfaces in the fight against microorganisms, especially bacteria and viruses. One of the benefits is the difficulty for these organisms to resist the multiple routes of attack against their forms of proliferation or replication, ensuring high efficiency and reduced possibility of developing microbiological resistance.

Especially with regard to antiviral activity, Galdiero et al. (2011, Doi: 10.3390/molecules16108894) describe in their review several routes of action that include interaction with glycoproteins present in the lipid layer of viruses and bacteria, disruption of viral cell and lipid membranes and/or blocking viral replication. DNA and RNA of microorganisms, through the interaction of antimicrobial agents directly with the target genome, or cellular/viral factors within cells.

PI 0011903-2 describes a fine paper product having an antiviral composition that includes a water-soluble film-forming vehicle or vehicles and one or more antiviral agents. Antiviral agents can include natural extracts, ascorbic acid and carboxylic acids. The fine paper product of said document may further include an optional moisture barrier and this may be treated with the antiviral composition.

Document BR 10 2015 031504-0 refers to a sponge antibacterial solution endowed with silver ions and its manufacturing process, which aims to inhibit the appearance of bacteria in the sponge that occurs according to use, conservation and packaging. There is also the addition of silver pigmentation with the functionality of representing the silver ions that will be inserted in the said sponge.

Therefore, it is possible to see that there are different solutions for blankets and sponges aimed at improving the cleaning, washing and scrubbing of surfaces of the most diverse materials, which may or may not include antimicrobial agents.

However, with the Covid-19 epidemic, it has become increasingly more necessary the care and cleaning of surfaces, whether domestic surfaces or hospital environments, taking into account the high transmission of the virus.

Thus, the present invention provides a blanket and a sponge with abrasive action that have greater durability throughout use, also comprising an antiviral and/or antibacterial agent. Thus, the blanket and sponge developed provide even more efficiency in combating different viruses, in addition to combating other microorganisms, pathogenic or not for humans and animals.

### SUMMARY OF THE INVENTIONS

A first object of the present invention is to provide a antiviral and/or antibacterial abrasive blanket that helps clean surfaces and fight pathogenic agents, inhibiting, neutralizing, destroying, reducing, killing or eliminating when present.

A second object of the present invention is to provide a process for producing said antiviral and/or antibacterial abrasive blanket.

A third objective of the present invention describes the use of antiviral and/or antibacterial abrasive blanket.

A fourth object of the present invention is to provide a antiviral and/or antibacterial cleaning sponge that, in addition to being used to clean surfaces, also helps to combat pathogenic agents, inhibiting, neutralizing, destroying, reducing, killing or eliminating when present.

A fifth objective of the present invention is to provide a process for producing said antiviral and/or antibacterial cleaning sponge.

Additionally, the present invention also describes the use antiviral and/or antibacterial cleaning sponge.

The purpose and other advantages of the present invention will be better evidenced from the detailed description that follows and the attached figure.

### BRIEF DESCRIPTION OF THE DRAWINGS

FIG. 1 identifies the stages of the production process of pre antiviral and/or antibacterial non-abrasive blanket.

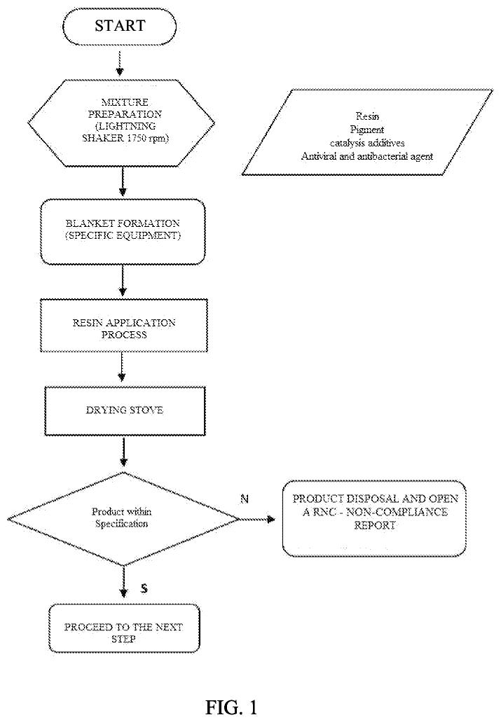

FIG. 2 identifies the next steps of the process started in FIG. 1, with the production of the abrasive and/or antibacterial blanket and the bonding of the abrasive blanket to the polyurethane foam.

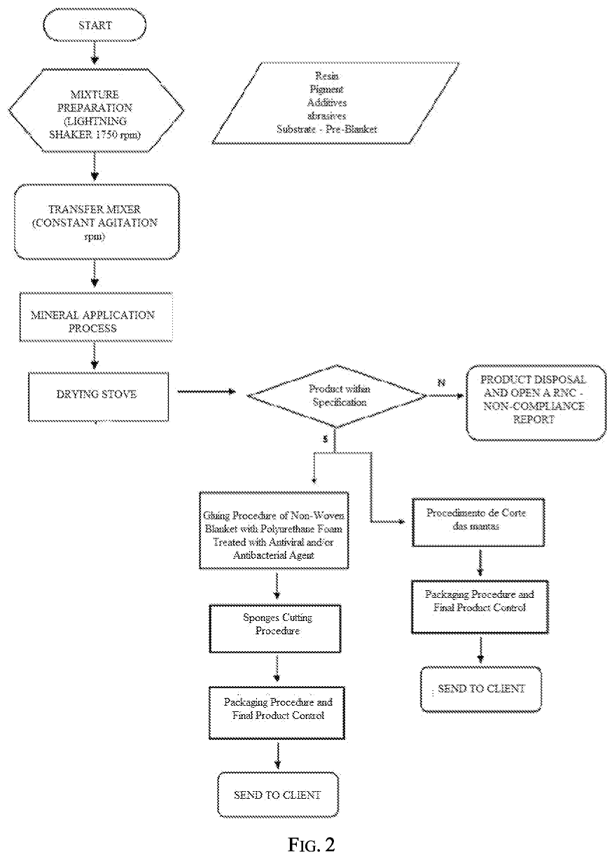

### DETAILED DESCRIPTION OF THE INVENTION

The present invention relates to an antiviral abrasive blanket and/or antibacterial for cleaning surfaces, also having microbicidal action against gram-positive and gram-negative bacteria, enveloped and non-enveloped viruses, as well as other microorganisms, such as fungi, mites, spores and the like, among other potentially pathogenic organisms for the human or animal.

The antiviral and/or antibacterial abrasive blanket of the present the invention acts by neutralizing, inhibiting, reducing, destroying, eliminating or killing said organisms due to their antiviral and/or antibacterial property.

More precisely, the antiviral abrasive blanket and/or antibacterial comprises a non-abrasive non-woven formed from synthetic and/or natural fibers and at least two polymeric emulsions,

wherein the non-abrasive non-woven is first soaked in a first polymeric emulsion comprising an antiviral and/or antibacterial mixture comprising at least one polymer, at least one pigment, at least one antiviral and/or antibacterial agent and optionally at least one solvent,

and then soaked in a second polymeric emulsion comprising an abrasive mixture comprising at least one polymer, at least one pigment, abrasive grains and optionally at least one solvent.

Non-Abrasive Non-Woven Fabric

For the present invention, the non-abrasive non-woven fabric of blanket refers to a product that has a flexible structure, preferably flat and porous, and may be made from fibers or filaments, with a predefined or random direction.

Among the different conventional processes for obtaining non-abrasive non-woven fabrics are chemical, mechanical, thermal processes, or a combination of two or more processes.

In one embodiment of the present invention, the non-abrasive non-woven fabric is formed from synthetic and/or natural fibers. Among the synthetic fibers, polyamide, polyester, or similar fibers or combinations thereof can be used, but not limited to. Natural fibers can be, among others, vegetable fibers, cotton, wool and or similar and their combinations.

First and Second Polymeric Emulsions

In the present invention, the first polymeric emulsion comprises an antiviral and/or antibacterial mixture which is responsible for adding the antiviral and/or antibacterial agent to the developed blanket, while the second emulsion comprises an abrasive mixture which is responsible for adding abrasive grains.

The first and second polymeric emulsions contain, in addition to antiviral and/or antibacterial agent or abrasive grains, polymers and pigments, and may optionally contain stabilizing and/or catalytic additives, as detailed below.

A) Antiviral and/or Antibacterial Mixture (First Polymeric Emulsion) Polymer

For the present invention, any thermoplastic or thermoset polymers can be used. Examples of useful polymers are, but are not limited to, acrylic, phenolic, vinyl, polyolefin, polyurethane polymers, or copolymers or the like or mixtures thereof.

In a preferred embodiment of the invention, the at least a polymer used is acrylic (co)polymer, such as polyacrylates, polymethacrylates, poly(methyl methacrylates), or a phenolic polymer.

In one embodiment of the invention, the first polymeric emulsion comprises from about 60% to about 95% of at least one polymer, more preferably from about 70% to about 90% of at least one polymer, and even more preferably from about 75% to about 90% of at least one polymer, based on the total weight of the first polymeric emulsion.

Stabilizing Additives

In the context of the present invention, stabilizing additives are the compounds that maintain the physical characteristics of suspensions and emulsions. These products can be thickeners, anti-foaming agents, preservatives, salts, co-surfactants, or similar and combinations thereof.

In one embodiment of the invention, the first polymeric emulsion comprises from about 0% to about 5% of at least one stabilizing additive, preferably from about 0.1% to about 4% of at least one stabilizing additive, and even more preferably from about 0.2% to about 3%, based on the total weight of the first polymeric emulsion.

In a preferred embodiment, the first emulsion antiviral and/or antibacterial polymeric comprises thickeners and/or antifoams.

Thickeners are products used to increase the viscosity of the emulsion. More precisely, in the case of an emulsion with acrylic polymers, thickeners for acrylic copolymers are used in the present invention.

Defoamers inhibit foaming in the polymer emulsion. Preferably, in the embodiment in which the antiviral and/or antibacterial polymeric emulsion is water-based, the antifoam employed is polydimethylsiloxane with auxiliary materials.

In an alternative embodiment, the first polymeric emulsion is free of stabilizing additives.

Pigments

In the present invention, the pigments are used for a better finish of the product, in addition to being able to be used for identification and differentiation based on specification.

In the present invention, the pigments can be blue, green, gray, yellow, red, orange, purple, pink, black but not limited to just these colors.

In one embodiment of the invention, about 0.1% to about 10% of at least one pigment is added, more preferably from about 1% to about 7% of at least one pigment, and even more preferably about 1% to about 5% of at least one pigment relative to the total weight of the first polymeric emulsion.

Catalysis Additives

Catalysis additives can be used to accelerate polymerization and give greater resistance to the blanket formed from the fibers of the pre-blanket, in addition to facilitating the process of combining the components of the first polymeric emulsion and the non-abrasive non-woven pre-blanket.

Also, catalysis additives are agents that help in the process of incorporating coatings, resulting in desired curing and finishing characteristics, which may or may not be heat-activated. Among the catalysis additives used in the present invention, amino or alkylated crosslinking additives can be mentioned preferably.

In one embodiment, the at least one catalyst additive is selected from alkylated urea, alkylated melamine formaldehyde, alkylated urea formaldehyde, benzoguanamine-formaldehyde, glycol uryl-formaldehyde, combinations or the like thereof. Preferably, the present invention uses methylated melamine-formaldehyde as catalyst additive.

Alternatively, the first polymeric emulsion is free of catalyst additives.

In one embodiment of the invention, about 0% to about 10% of at least one catalysis additive, preferably from about 1% to about 9% of at least one catalysis additive, and more preferably about from 2% to about 8% of at least one catalyst additive, based on the total weight of the first polymeric emulsion.

Antiviral and/or Antibacterial Agent

In the present invention, the at least one antiviral agent and/or antibacterial is responsible for the elimination of bacteria, viruses, and may also inhibit, neutralize, destroy, reduce, kill or eliminate other microorganisms such as, but not limited to, fungi, mites and spores.

In general, viruses can be enveloped or not. In both cases, these microorganisms are made up of a genome associated with basic proteins. This structure (genome+basic proteins) is called the nucleus. External to the nucleus is the capsid, which is a protein layer that covers the viral genome. The nucleus plus the capsid is called the nucleus capsid, which, in enveloped viruses, is again covered by a lipoprotein membrane called the envelope, thus giving it the characteristic of being enveloped. This bilipid layer often has the characteristic of the virus host cell.

Non-enveloped viruses do not have the bilipid layer for additional protection described above, only the protective protein capsid. Due to this, its structure is usually referred to as “more resistant”, as its genetic code directly supports factors such as acidity, temperature and biological agents to persist in the environment.

Bacteria, on the other hand, can be gram-positive or gram-negative. Gram-positive bacteria have a single-layered cell wall while gram-negative bacteria have a cell wall made up of multi-layered structures. Thus, gram-negative bacteria are more resistant than gram-positive ones. In addition, gram-negative bacteria are more virulent than gram-positive bacteria.

In one embodiment of the present invention, the at least one antiviral and/or antibacterial agent is selected from nanoparticles with a nucleus composed of metallic atoms, in which such nanoparticles may or may not be stabilized with organic molecules.

With respect to the average size of the nanoparticles, at least one of its dimensions is from about 1 to about 100 nm, more preferably from about 1 to about 75 nm, and even more preferably from about 1 to about 50 nm.

The metallic atoms of the nuclei of nanoparticles can be selected from copper, silver, gold, zinc, or similar or combinations thereof. When stabilized with organic molecules, such molecules are preferably selected from monomers, polymers, polyphenols, polyflavonoids, organic acids, silanes, siloxanes, or similar or combinations thereof.

In an alternative embodiment of the invention, the at least one antiviral and/or antibacterial agent comprises organic molecules (organic antimicrobial compounds) containing active principles that include, but are not limited to, ammonium quaternaries, cationic polysaccharides (such as chitosan), saturated dialdehydes (as glutaraldehyde), isothiazolinones, pure or in mixtures, their like and combinations thereof.

In one embodiment of the invention, the antiviral and/or antibacterial polymeric emulsion comprises about 0.1% to about 10% of at least one antiviral and/or antibacterial agent, preferably about 0.5% to about 5% of at least one antiviral and/or antibacterial agent, and even more preferably about 1.5% to about 3% of at least one antiviral and/or antibacterial agent, relative to the total weight of the first polymeric emulsion.

Solvent

With regard to the solvent used, if necessary, to form the first polymeric emulsion, any organic or inorganic solvents may be used such as, but not limited to, water, a glycol, an alcohol, another polar solvent, and combinations thereof. Preferably, the solvent used in the present invention is water.

In the present invention, the at least one solvent, if present, is added to the first polymeric emulsion in q.s.p (a sufficient quantity) 100% by weight, based on the total weight of the first polymeric emulsion. Preferably, the at least one added solvent amounts to about 0.1% to about 10% by weight of the total first polymeric emulsion. More preferably, the at least one added solvent amounts to about 5% by weight of the total first polymeric emulsion.

B) Abrasive Mixture (Second Polymeric Emulsion) Polymer

For the present invention, any thermoplastic or thermoset polymers can be used. Examples of useful polymers are, but are not limited to, acrylic, phenolic, vinyl, polyolefin, polyurethane polymers, or copolymers or the like or mixtures thereof.

In a preferred embodiment of the invention, the at least a polymer used is acrylic (co)polymer, such as polyacrylates, polymethacrylates, poly (methyl methacrylates), or a phenolic polymer.

In an embodiment of the invention, the second abrasive polymeric emulsion comprises from about 10% to about 50% of at least one polymer, more preferably from about 20% to about 40% of at least one polymer and even more preferably about 30% of at least one polymer, in relation to the total weight of the second polymeric emulsion.

Stabilizing Additives

In the context of the present invention, stabilizing additives are as described above.

In a preferred embodiment, the second polymeric emulsion comprises thickeners and/or defoamers. Alternatively, the second polymeric emulsion can be free of such additives.

In one embodiment of the invention, the polymeric emulsion abrasive comprises from about 0% to about 5% of at least one stabilizing additive, preferably from about 0.1% to about 4% of at least one stabilizing additive, and even more preferably about 0.2% to about of 3%, in relation to the total weight of the second polymeric emulsion.

Pigments

In the present invention, the pigments used are as previously described.

In one embodiment of the invention, about 0.1% to about 10% of at least one pigment is added, more preferably from about 1% to about 7% of at least one pigment, and even more preferably about 1.5% of at least one pigment in relation to the total weight of the second polymeric emulsion.

The catalysis additives of the second polymeric emulsion are as previously defined.

In a preferred embodiment, the catalyst additive used is methylated melamine-formaldehyde. In an alternative embodiment, the second polymeric emulsion is free of catalysis additives.

In one embodiment of the invention, about 0% to about 10% of at least one catalysis additive, preferably from about 1% to about 9% of at least one catalysis additive, and more preferably about from 2% to about 8% of at least one catalyst additive, based on the total weight of the second polymeric emulsion.

Abrasive Grains

Abrasive grains are substances of natural or synthetic origin used for surface treatment by means of cleaning, grinding, leveling and polishing.

In one embodiment of the invention, the abrasive grains used can be any natural or synthetic grains such as, but not limited to, garnet, emery, flint, oxides and/or and/or carbides and/or metal nitrides, preferably oxides of Ma or IVa families of the periodic table, ceramic oxides and their combinations. For example, abrasive grains can include, among other components, silica, alumina, zirconia, silicon carbide, boron carbide, titanium carbide, titanium dioxide, iron oxide, tin oxide, aluminum oxide or combinations thereof. Preferably, aluminum oxide is used.

It is possible to have grains of different shapes, which include rod, cylinder, cone, triangle, solid or hollow sphere, or similar and random combinations of different sizes and shapes.

In one embodiment of the invention, the second emulsion polymer comprises about 40% to about 80% abrasive grains, preferably about 50% to about 70% abrasive grains, and even more preferably about 55% to about 65% abrasive grains, by total weight of the second polymeric emulsion.

Solvent

With regard to the solvent used, if necessary, to form the second polymeric emulsion, the previously described solvents can be used.

In the present invention, the at least one solvent, if present, is added to the second polymeric emulsion in q.s.p (a sufficient quantity) 100% by weight, based on the total weight of the second polymeric emulsion. Preferably, the at least one added solvent amounts to about 0.1% to about 10% by weight of the total second polymeric emulsion. More preferably, the at least one added solvent amounts to about 5% by weight of the total second polymeric emulsion.

The present invention also relates to a process for manufacture of the antiviral and/or antibacterial abrasive blanket, comprising the steps of:
(I) forming a non-abrasive non-woven antiviral and/or antibacterial blanket comprising the steps of:
   form a non-abrasive non-woven pre-blanket with synthetic and/or natural fibers,
   separately to the formation of the pre-blanket, prepare an antiviral and/or antibacterial mixture in emulsion comprising the steps of:
      optionally mixing at least one stabilizing additive, at least one polymer, and optionally at least one solvent, under agitation, forming a first polymeric emulsion,
      adding at least one pigment and optionally at least one catalyst additive to the first polymeric emulsion under agitation,
      adding at least one antiviral and/or antibacterial agent to the first polymeric emulsion under agitation, forming the antiviral and/or antibacterial mixture into an emulsion,
      after homogenizing the antiviral and/or antibacterial mixture in emulsion, keep stirring for at least about 10 minutes,
   incorporate the antiviral and/or antibacterial emulsion mixture into the non-abrasive non-woven pre-blanket,
   perform a first curing step on the pre-blanket incorporated with the antiviral and/or antibacterial mixture at least about 100° C. at an air speed of about 5 m/s to about 10 m/s, forming an antiviral blanket and/or antibacterial;
(II) forming an antiviral and/or antibacterial abrasive blanket comprising the steps of:
   preparing an emulsion abrasive mixture comprising the steps of:
      optionally mixing at least one stabilizing additive, at least one polymer, and optionally at least one solvent, under stirring, forming a second polymeric emulsion,
      adding at least one pigment and optionally at least one catalysis additive to the second polymeric emulsion under agitation,
      add abrasive grains to the second polymeric emulsion under agitation, forming an abrasive mixture in emulsion;
      after homogenizing the abrasive mixture into an emulsion, keep stirring for at least about 10 minutes,
   incorporate the abrasive mixture in emulsion into the non-abrasive non-woven antiviral and/or antibacterial blanket,
   performing a second curing step on the antiviral and/or antibacterial blanket incorporated with the abrasive mixture at least about 100° C. at an air speed of about 5 m/s to about 10 m/s.

In the present invention, synthetic and/or natural fibers and first to second polymeric emulsions are as detailed in the preceding paragraphs.

In one embodiment of the invention, at least a third addition of a polymeric emulsion comprising an antiviral and/or antibacterial mixture or abrasive mixture, or a combination thereof, followed by at least a third curing step may be made.

In an alternative embodiment of the invention, the pre-blanket of non-woven material can be initially soaked with the abrasive mixture in emulsion and then cured, forming a cured abrasive blanket, and then said cured abrasive blanket can be soaked with the antiviral and/or antibacterial mixture, going through a second process of cure.

In one embodiment of the invention described herein, agitation employed in the process steps ranges from about 1000 rpm to about 2000 rpm, more preferably from about 1750 rpm.

In one embodiment of the invention, post-homogenization agitation of the abrasive mixture or antiviral and/or antibacterial mixture is maintained for at least about 10 minutes, preferably for about 10 minutes to about 30 minutes, and more preferably for about 20 minutes. minutes.

In the present invention, the stages of incorporation of the mixtures in emulsion (antiviral and/or antibacterial mixture and abrasive mixture) in the pre-blanket and in the antiviral and/or antibacterial blanket occurs by a transfer roller set system or by spraying (spray). In a preferred embodiment, the incorporation of the antiviral and/or antibacterial mixture in emulsion into the pre-blanket takes place by a transfer roller assembly system, while the incorporation of the abrasive mixture in emulsion into the antiviral and/or antibacterial blanket occurs by spraying.

In one embodiment of the invention, the first and second curing steps of the web manufacturing process are carried out in an oven at least about 100° C., preferably at about 100° C. to about 250° C., and even more preferably at 180° C. at an air velocity of from about 5 m/s to about 10 m/s, preferably from about 6 m/s to about 8 m/s and even more preferably at 7.5 m/s.

The present invention also refers to the use of a blanket antiviral and/or antibacterial abrasive as described above.

More specifically, the abrasive blanket can be used in soft or hard surfaces. Preferably, the abrasive blanket of the present invention is used on surfaces selected from, but not limited to, floors or coatings, tiles, leather, clothing, fabrics or non-woven, tableware and household utensils, or similar products.

More precisely, the antiviral abrasive blanket and/or antibacterial can be used in the disinfection of hospitals, veterinary clinics, household cleaning in public or private buildings, clothing, fabrics and non-woven, vehicles, agricultural production utensils, animal production, cleaning sewage collection systems, washing fresh products (meats, fruits, vegetables, etc.), or cleaning areas and equipment in food processing.

Additionally, the antiviral and/or antibacterial abrasive blanket can be used alone or in conjunction with another support material on one of its faces. For example, the antiviral and/or antibacterial abrasive blanket can be used together with a foam, thus forming a sponge in which one side is the abrasive blanket, while the opposite side is the foam.

The present invention also relates to a sponge of antiviral and/or antibacterial wipe, comprising a first side and a second side,

wherein the first side comprises a foam optionally comprising an antiviral and/or antibacterial agent and the second side comprises a non-woven antiviral and/or antibacterial abrasive blanket,

wherein the non-woven antiviral and/or antibacterial abrasive blanket comprises natural and/or synthetic fibers and at least two polymeric emulsions,

wherein the non-woven is first soaked in a first polymeric emulsion comprising an antiviral and/or antibacterial blend comprising at least one polymer, at least one pigment, at least one antiviral and/or antibacterial agent, and optionally at least one solvent,

and then soaked in a second polymeric emulsion comprising an abrasive mixture comprising at least one polymer, at least one pigment, abrasive grains and optionally at least one solvent.

In the present invention, synthetic and/or natural fibers, and first and second polymeric emulsions are as detailed in the preceding paragraphs.

Foam

In the present invention, the foam is composed of at least a flexible natural or synthetic polymer such as polyurethane or cellulose-based foam. In a preferred embodiment, the foam is a polyurethane foam.

In one embodiment of the invention, the foam can comprise up to about 15% of at least one antiviral and/or antibacterial agent, based on the total weight of its composition. Preferably, the foam comprises from about 0.1% to about 5% of at least one antiviral and/or antibacterial agent, and more precisely about 1.5% to about 3% of at least one antiviral and/or antibacterial agent or antibacterial. At least one antiviral and/or antibacterial agent is as previously listed.

The present invention also relates to a process for manufacture of antiviral and/or antibacterial cleaning sponge, comprising stages of:
(I) forming a non-abrasive non-woven antiviral and/or antibacterial blanket comprising the steps of:
   form a non-abrasive non-woven pre-blanket with synthetic and/or natural fibers,
   separately to the formation of the pre-blanket, prepare an antiviral and/or antibacterial mixture in emulsion comprising the steps of:
      optionally mixing at least one stabilizing additive, at least one polymer, and optionally at least one solvent, under stirring, forming a first polymeric emulsion,
      adding at least one pigment and optionally at least one catalysis additive to the first polymeric emulsion under agitation,
      adding at least one antiviral and/or antibacterial agent to the first polymeric emulsion under agitation, forming the antiviral and/or antibacterial mixture into an emulsion,
      after homogenizing the antiviral and/or antibacterial mixture in emulsion, keep stirring for at least about 10 minutes,
   incorporate the antiviral and/or antibacterial emulsion mixture into the non-abrasive non-woven pre-blanket,
   perform a first curing step on the pre-blanket incorporated with the antiviral and/or antibacterial mixture at least about 100° C. at an air speed of about 5 m/s to about 10 m/s, forming an antiviral blanket and/or antibacterial;
(II) form an antiviral and/or antibacterial abrasive blanket comprising the steps of:
   preparing an emulsion abrasive mixture comprising the steps of:
      optionally mixing at least one stabilizing additive, at least one polymer, and optionally at least one solvent, under stirring, forming a second polymeric emulsion,
      adding at least one pigment and optionally at least one catalysis additive to the second polymeric emulsion under agitation,
      add abrasive grains to the second polymeric emulsion under agitation, forming an abrasive mixture in emulsion,
      after homogenizing the abrasive mixture into an emulsion, keep stirring for at least about 10 minutes,
      incorporate the abrasive mixture in emulsion into the non-abrasive non-woven antiviral and/or antibacterial blanket,
      performing a second curing step on the antiviral and/or antibacterial blanket incorporated with the abrasive mixture at least about 100° C. at an air speed of about 5 m/s to about 10 m/s,
(III) bonding the antiviral and/or antibacterial abrasive blanket to a foam which optionally comprises an antiviral and/or antibacterial agent.

In the present invention, synthetic and/or natural fibers and first and second emulsions are as detailed in the preceding paragraphs.

In one embodiment of the invention, at least a third addition of a polymeric emulsion comprising an antiviral and/or antibacterial mixture or abrasive mixture, or a combination thereof, followed by at least a third curing step.

In an alternative embodiment of the invention, the pre-blanket of non-woven material can be initially soaked with the abrasive mixture in emulsion and then cured, forming a cured abrasive blanket, and then said cured abrasive blanket can be soaked with the antiviral and/or antibacterial mixture, going through a second process of cure.

In an embodiment of the invention described herein, agitation employed in the process steps ranges from about 1000 rpm to about 2000 rpm, more preferably from about 1750 rpm.

In one embodiment of the invention, the post-homogenization agitation of the abrasive mixture or antiviral and/or antibacterial emulsion mixture is maintained for at least about 10 minutes, preferably for about 10 minutes to about 30 minutes, and more preferably, for about 20 minutes.

In the present invention, the incorporation steps of the emulsion mixtures (antiviral and/or antibacterial mixture and abrasive mixture) in the pre-blanket and in the antiviral and/or antibacterial blanket occur by a transfer roller set system or by spraying (spray). In a preferred embodiment, the incorporation of the antiviral and/or antibacterial mixture in emulsion into the pre-blanket takes place by a transfer roller assembly system, while the incorporation of the abrasive mixture in emulsion into the antiviral and/or antibacterial blanket occurs by spraying.

In one embodiment of the invention, the first and second curing steps of the sponge manufacturing process are carried out in an oven at least about 100° C., preferably at about 100° C. to about 250° C., and even more preferably at 180° C. at an air velocity of from about 5 m/s to about 10 m/s, preferably from about 6 m/s to about 8 m/s and even more preferably at 7.5 m/s.

In the gluing step, the union between the foam and the blanket antiviral and/or antibacterial abrasive is made by means of an adhesive agent.

Adhesive Agent

Adhesive agents are used to join at least two surfaces. Adhesive agents are solvent-based, water-based, hot melt adhesives, pressure sensitive adhesives, among others.

In the present invention, as adhesive agents, any adhesives based on epoxy, silicone, polyurethane and combinations thereof can be used. Preferably, the adhesive used in the present invention is a solvent-free polyurethane adhesive.

Additionally, the foam may optionally comprise an antiviral and/or antibacterial agent, as previously described.

Also, after the gluing step, the cleaning sponge produced is cut into predetermined formats for commercialization.

The present invention further relates to the use of an antiviral and/or antibacterial cleaning sponge as described above, wherein its use is similar to the previously described use of the antiviral and/or antibacterial abrasive blanket.

The description that follows will start from an embodiment particular of the invention. As will be apparent to anyone skilled in the art, the invention is not limited to such an embodiment.

### EXAMPLE 1—PRODUCTION OF ANTIVIRAL AND/OR ANTIBACTERIAL ABRASIVE BLANKET

The production process of the abrasive blanket starts with the preparation of the antiviral and/or antibacterial mixture (first polymeric emulsion) in a lightning shaker (industrial mixer) with a stirring speed of approximately 1750 rpm.

In the lightning stirrer, the polydimethylsiloxane and auxiliary materials are added under agitation in a concentration of approximately 3%, in relation to the total weight of the first polymeric emulsion, and a mixture containing approximately 84% of acrylic polymers and about 4% of water, also in relation to the total weight of the first water-based emulsion.

Then, a green pigment is added to the mixture at a concentration of about 1.5%, in relation to the total weight of the first polymeric emulsion. Still in this step, formaldehyde-alkylated melamine is also added as a catalyst additive in a concentration of about 5%, in relation to the total weight of the first polymeric emulsion.

In the next step, nanoparticles with a nucleus composed of silver atoms are added at a concentration of about 2.5%, in relation to the total weight of the first polymeric emulsion, forming an antiviral and/or antibacterial mixture in emulsion. This emulsion mixture is then homogenized and kept stirring for about 20 minutes.

At the same time, a non-abrasive non-woven pre-blanket is produced from polyamide/polyester fibers, in a non-abrasive non-woven batt forming machine.

Then, the prepared antiviral and/or antibacterial emulsion mixture is applied to the pre-blanket through a transfer roller assembly system. The pre-blanket incorporated with the emulsion mixture is then sent to a drying oven, where curing takes place at approximately 180° C. with an air speed of about 7.5 m/s, forming an antiviral and/or antibacterial blanket.

After the curing stage, the antiviral and/or antibacterial blanket undergoes a quality test. If the product is out of specification, the defective product is discarded and a nonconformity report is filled out. If the product is within specification, it is sent to the abrasive grain application stage.

In parallel, a second polymeric emulsion is produced. In a lightning shaker, the polydimethylsiloxane and auxiliary materials are added under agitation in a concentration of approximately 0.2%, in relation to the total weight of the second polymeric emulsion, and a mixture containing approximately 29% of acrylic polymers and about 7% of water, also in relation to the total weight of the second water-based emulsion.

Then, a green pigment is added to the mixture at a concentration of about 1.8%, based on the total weight of the second polymeric emulsion. Also in this step, formaldehyde-alkylated melamine is added as a catalyst additive at a concentration of about 2%, in relation to the total weight of the second polymeric emulsion.

Then, abrasive grains are added at a concentration of approximately 60% in relation to the total weight of the second polymeric emulsion, forming an abrasive mixture in emulsion. This emulsion mixture is then homogenized and kept stirring for 20 minutes.

The abrasive mixture in emulsion is incorporated into the antiviral and/or antibacterial blanket by spraying and a new curing step is carried out under conditions equivalent to those of the first curing step, producing an abrasive antiviral and/or antibacterial blanket.

The product can be packaged and passed through a final control to be sent to the customer or sent to a process for the production of a cleaning sponge.

### EXAMPLE 2—PRODUCTION OF ANTIVIRAL AND/OR ANTIBACTERIAL CLEANING SPONGE

The antiviral cleaning sponge production process begins with the preparation of the antiviral and/or antibacterial mixture (first polymeric emulsion) in a lightning shaker (industrial mixer) with a stirring speed of approximately 1750 rpm.

In the lightning stirrer, a mixture containing approximately 87.5% of phenolic resin and approximately 8% of water, relative to the total weight of the first water-based emulsion, is added under agitation.

Then, a green pigment is added to the mixture at a concentration of about 1.5%, based on the total weight of the first polymeric emulsion.

In the next step, nanoparticles with a nucleus composed of silver atoms are added in a concentration of about 3%, in relation to the total weight of the first polymeric emulsion, forming an antiviral and/or antibacterial mixture in emulsion. This emulsion mixture is then homogenized and kept stirring for about 20 minutes.

At the same time, a non-abrasive non-woven pre-blanket is produced from polyamide/polyester fibers, in a non-abrasive non-woven batt forming machine.

Then, the prepared antiviral and/or antibacterial emulsion mixture is applied to the pre-blanket through a transfer roller assembly system. The pre-blanket incorporated with the emulsion mixture is then sent to a drying oven, where curing takes place at approximately 180° C. with an air speed of about 7.5 m/s, forming an antiviral and/or antibacterial blanket.

After the curing step, the antiviral and/or antibacterial blanket passes a quality test. If the product is out of specification, the defective product is discarded and a nonconformity report is filled out.

After the first cure and quality test, the antiviral and/or antibacterial blanket is sent to the abrasive grain application stage.

In parallel, the second polymeric emulsion is produced. In a lightning stirrer, a mixture containing approximately 30% of phenolic polymers and approximately 8% of water, based on the total weight of the second water-based emulsion, is added under agitation.

Next, a green pigment is added to the mixture in a concentration of about 1.5%, based on the total weight of the second polymeric emulsion.

Then, abrasive grains are added at a concentration of approximately 60.5% in relation to the total weight of the second polymeric emulsion, forming an abrasive mixture in emulsion. This emulsion mixture is then homogenized and kept stirring for 20 minutes.

The abrasive mixture in emulsion is incorporated in the blanket antiviral and/or antibacterial by spraying and a new curing step is carried out under conditions equivalent to those of the first curing step, producing an abrasive antiviral and/or antibacterial blanket.

Once produced, the antiviral and/or antibacterial abrasive blanket is sent to a bonding step.

In that step, a soft polyurethane foam is bonded to the abrasive blanket using a solvent-free polyurethane adhesive.

The final product is then cut into parallelepipeds and packaged, and may also undergo final control for shipment to the customer.

In this way, both the abrasive blanket and the antiviral and/or antibacterial cleaning sponge developed in the present invention promote benefits for the health and well-being of consumers, due to the antiviral and antibacterial action, which fights viruses and bacteria that are lodged in these utensils. and replicate quickly, and can be harmful to individuals.

In addition, because the blanket and sponge are protected against these microorganisms, the product is no longer a means of contamination for viruses and bacteria and, consequently, prevents cross-contamination between surfaces, utensils and environments that will be cleaned and sanitized. with the abrasive blanket or the antiviral and/or antibacterial cleaning sponge.

In the event of epidemics and pandemics such as the COVID-19 epidemic (SARS COV-2), cleaning and maintenance of environments and surfaces are essential. In addition, increasing the viability and durability of cleaning tools is necessary, which reduces the need for quick disposal/replacement of the same.

Thus, the production processes of the abrasive blanket and the cleaning sponge, in which there are at least two curing steps, with the antiviral and/or antibacterial agent being added and cured in one step, while the abrasive agent is added and cured in another step, result in products with greater durability and efficiency. This is because the processes of the present invention enhance the retention of both the antiviral and/or antibacterial agent and the abrasive agent, preventing their leaching onto the surfaces to be cleaned throughout use.

Countless variations affecting the scope of protection of the present application are allowed. In this way, it reinforces the fact that the present invention is not limited to the particular configurations/embodiments described above.

1. Antiviral and/or antibacterial abrasive blanket, characterized in that it comprises a non-abrasive nonwoven formed from synthetic and/or natural fibers and at least two polymeric emulsions,
wherein the non-abrasive nonwoven is soaked in a first polymeric emulsion comprising an antiviral and/or antibacterial mixture comprising;
about 60% to about 95% of at least one polymer,
about 0.1% to about 5% of at least one stabilizing additive,
about 0.1% to about 10% of at least one pigment,
about 1% to about 10% of at least one catalyst additive,
about 0.1% to about 10% of at least one antiviral and/or antibacterial agent, and
optionally at least one solvent in q.s.p (a sufficient quantity) 100% by weight,
the concentrations being relative to the total weight of the first
polymeric emulsion
and
it is then soaked in a second polymeric emulsion comprising an abrasive mixture comprising
about 10% to about 50% of at least one polymer,
about 0.1% to about 5% of at least one stabilizing additive,
about 0.1% to about 10% of at least one pigment,
about 1% to about 10% of at least one catalyst additive,
about 40% to about 80% of abrasive grains, and
optionally at least one solvent in q.s.p (a sufficient quantity) 100% by weight,
the concentrations being relative to the total weight of the second
polymeric emulsion.
at least one polymer, at least one pigment, abrasive grains and optionally at least one solvent.

2. Antiviral and/or antibacterial abrasive blanket according to claim 1, characterized in that the nonwoven fibers are synthetic fibers selected from polyamide, polyester, their similar and their mixtures.

3. Antiviral and/or antibacterial abrasive blanket according to claim 1 or 2, characterized in that the at least one polymer is selected from acrylic, phenolic, vinyl, polyolefins, polyurethanes, or their copolymers or similar or your mixtures.

4. Antiviral and/or antibacterial abrasive blanket according to any one of claims 1 to 3, characterized in that the at least one stabilizing additive is selected from thickeners, antifoams, preservatives, salts, co-surfactants, or similar and their combinations, being preferably selected from thickeners and anti-foaming agents.

5. Antiviral and/or antibacterial abrasive blanket according to any one of claims 1 to 4, characterized in that the at least one catalysis additive is selected from alkylated urea, alkylated melamine-formaldehyde, alkylated urea-formaldehyde, benzoguanamine-formaldehyde, glycol uryl-formaldehyde or the like and combinations thereof, preferably being methylated melamine-formaldehyde.

6. Antiviral and/or antibacterial abrasive blanket according to any one of claims 1 to 5, characterized in that the at least one antiviral and/or antimicrobial agent is selected from nanoparticles with a core composed of metallic atoms and/or organic molecules comprising ammonium quaternaries, cationic polysaccharides, saturated dialdehydes, isothiazolinones, pure or in mixtures, the like and combinations thereof,
wherein at least one dimension of the nanoparticles having a core composed of metal atoms is from about 1 nm to about 100 nm and the metal atoms are selected from copper, silver, gold, zinc or the like or combinations thereof, and
in which the nanoparticles can be stabilized with organic molecules selected from monomers, polymers, polyphenols, polyflavonoids, organic acids, silanes, siloxanes, or similar and combinations thereof.

7. Antiviral and/or antibacterial cleaning sponge, characterized in that it comprises a first side and a second side,
wherein the first side comprises a foam optionally comprising an antiviral and/or antibacterial agent and the second side comprises an abrasive antiviral and/or antibacterial blanket. or non-woven antibacterial,
wherein the non-woven antiviral and/or antibacterial abrasive blanket comprises natural and/or synthetic fibers and at least two polymeric emulsions,
wherein the non-woven is first soaked in a first polymeric emulsion comprising an antiviral and/or antibacterial blend comprising:
about 60% to about 95% of at least one polymer,
about 0.1% to about 5% of at least one stabilizing additive,
about 0.1% to about 10% of at least one pigment,
about 1% to about 10% of at least one catalyst additive,
about 0.1% to about 10% of at least one antiviral
and/or antibacterial agent, and
optionally at least one solvent in q.s.p (a sufficient quantity)
100% by weight,
the concentrations being relative to the total weight of the first polymeric emulsion,
and
wherein the second polymeric emulsion comprising the abrasive mixture comprises:
about 10% to about 50% of at least one polymer,
about 0.1% to about 5% of at least one stabilizing additive,
about 0.1% to about 10% of at least one pigment,
about 1% to about 10% of at least one catalyst additive,
about 40% to about 80% abrasive grains, and
optionally at least one solvent in q.s.p (a sufficient quantity) 100% by weight,
the concentrations being in relation to the total weight of the second polymeric emulsion
at least one catalysis additive.

8. Antiviral and/or antibacterial cleaning sponge according to claim 7, characterized in that the nonwoven fibers are synthetic fibers selected from polyamide, polyester, the like and mixtures thereof.

9. Antiviral and/or antibacterial cleaning sponge according to claim 7 or 8, characterized in that the at least one polymer is selected from acrylic polymers, vinyl phenolics, polyolefins, polyurethanes, or their copolymers or similar or mixtures thereof.

10. Antiviral and/or antibacterial cleaning sponge accordingly with any one of claims 7 to 9, characterized in that the at least one stabilizing additive is selected from thickeners, anti-foaming agents, preservatives, salts, co-surfactants, or the like and combinations thereof, preferably being selected from thickeners and anti-foam.

11. Antiviral and/or antibacterial cleaning sponge according to any one of claims 7 to 10, characterized in that the at least one catalyst additive is selected from alkylated urea, alkylated melamine-formaldehyde, alkylated urea-formaldehyde, benzoguanamine-formaldehyde, glycol uryl-formaldehyde, or similar and combinations thereof, preferably being methylated melamine-formaldehyde.

12. Antiviral and/or antibacterial cleaning sponge according to any one of claims 7 to 11, characterized in that the at least one antiviral and/or antimicrobial agent is selected from nanoparticles with a core composed of metal atoms and/or molecules organic compounds comprising ammonium quaternaries, cationic polysaccharides, saturated dialdehydes, isothiazolinones, pure or in mixtures, their similar and combinations thereof,
wherein at least one dimension of the nanoparticles having a
core composed of metal atoms is from about 1 nm to about 100 nm and the metal atoms are selected from copper, silver, gold, zinc or the like or combinations thereof, and
in which the nanoparticles can be stabilized with organic molecules selected from monomers, polymers, polyphenols, polyflavonoids, organic acids, silanes, siloxanes, or similar and combinations thereof.

13. Antiviral and/or antibacterial cleaning sponge according to any one of claims 7 to 12, characterized in that the foam is a polyurethane or cellulose-based foam, preferably a polyurethane foam.

14. Process for manufacturing an antiviral and/or antibacterial abrasive blanket, characterized in that it comprises the steps of:
(I) formar uma manta antiviral e/ou antibacteriana de não tecido não abrasivo que compreende as etapas de:
form a non-abrasive non-woven pre-blanket with synthetic and/or natural fibers,
separately to the formation of the pre-blanket, prepare an antiviral and/or antibacterial mixture in emulsion comprising the steps of:
optionally mixing about 0.1% to about 5% of at least one stabilizing additive, about 60% to about 95% of at least one polymer, and optionally at least one solvent in q.s.p (a sufficient quantity) 100% by weight, under stirring, forming a first polymeric emulsion,
adding about 0.1$ to about 10% of at least one pigment and optionally about 1% to about 10% of at least one catalysis additive to the first polymeric emulsion under agitation,
adding about 0.1% to about 10% of at least one antiviral and/or antibacterial agent to the first polymeric emulsion under agitation, forming the antiviral and/or antibacterial mixture into an emulsion, the concentrations being relative to the total weight of the first polymeric emulsion.
after homogenizing the antiviral and/or antibacterial mixture in emulsion, keep stirring for at least about 10 minutes,
incorporate the antiviral and/or antibacterial emulsion mixture into the non-abrasive non-woven pre-blanket,
perform a first curing step on the pre-blanket incorporated with the antiviral and/or antibacterial mixture at least about 100° C. to about 250° C. at an air speed of about 5 m/s to about 10 m/s, forming an antiviral blanket and/or antibacterial;
(II) form the antiviral and/or antibacterial abrasive blanket comprising the steps of:
preparing an emulsion abrasive mixture comprising the steps of:
optionally mixing about 0.1% to about 5% of at least one stabilizing additive, about 10% to about 50% of at least one polymer,
and optionally at least one solvent in q.s.p (a sufficient quantity) 100% by weight, under stirring, forming a second polymeric emulsion,
adding about 0.1% to about 10% of at least one pigment and optionally about 1% to about 10% of at least one catalysis additive to the second polymeric emulsion under agitation,
add about 40% to about 80% of abrasive grains to the second polymeric emulsion under agitation, forming an abrasive mixture in emulsion, the concentrations being relative to the total weight of the second polymeric emulsion.
after homogenizing the abrasive mixture into an emulsion, keep stirring for at least about 10 minutes,
incorporate the abrasive mixture in emulsion into the non-abrasive non-woven antiviral and/or antibacterial blanket,
performing a second curing step on the antiviral and/or antibacterial blanket incorporated with the abrasive mixture at least about 100° C. to about 250° C. at an air speed of about 5 m/s to about 10 m/s.

15. Process according to claim 14, characterized in that the stirring is from about 1000 rpm to about 2000 rpm, and in which the post-homogenization stirring of the emulsion mixtures is maintained for about 10 to about 30 minutes, preferably about 20 minutes.

16. Process according to claim 14 or 15, characterized in that the first and second curing steps are carried out at 180° C. at an air velocity of about 6 m/s to about 8 m/ss.

17. Process according to any one of claims 14 to 16, characterized in that the fibers of the non-woven pre-blanket are synthetic fibers selected from polyamide, polyester, or similar fibers or combinations thereof.

18. Process according to any one of claims 14 to 17, characterized in that the first and second polymeric emulsions additionally comprise at least one stabilizing additive and/or at least one catalysis additive.

19. Process according to any one of claims 14 to 18, characterized in that the at least one polymer is selected from acrylic, phenolic, vinyl, polyolefins, polyurethanes, or their copolymers or similar or mixtures thereof.

20. Process according to any one of claims 14 to 19, characterized in that the at least one stabilizing additive is selected from thickeners, anti-foaming agents, preservatives, salts, co-surfactants, or similar and combinations thereof, being preferably selected from thickeners and anti-foaming agents.

21. Process according to any one of claims 14 to 20, characterized in that the at least one catalysis additive is selected from alkylated urea, alkylated melamine-formaldehyde, alkylated urea-formaldehyde, benzoguanamine-formaldehyde, uryl glycol-formaldehyde, or similar and combinations thereof, preferably methylated melamine-formaldehyde.

22. Process according to any one of claims 14 to 21, characterized in that the at least one antiviral and/or antimicrobial agent is selected from nanoparticles with a nucleus composed of metallic atoms and/or organic molecules comprising ammonium quaternaries, cationic polysaccharides, saturated dialdehydes, isothiazolinones, pure or in mixtures, their similar and combinations thereof,
wherein at least one dimension of the nanoparticles having a core composed of metal atoms is from about 1 nm to about 100 nm and the metal atoms are selected from copper, silver, gold, zinc or the like or combinations thereof, and
in which the nanoparticles can be stabilized with organic molecules selected from monomers, polymers, polyphenols, polyflavonoids, organic acids, silanes, siloxanes, or similar and combinations thereof.

23. Process for manufacturing an antiviral and/or antibacterial cleaning sponge, characterized in that it comprises the steps of:
(I) forming a non-abrasive non-woven antiviral and/or antibacterial blanket comprising the steps of:
form a non-abrasive non-woven pre-blanket with synthetic and/or natural fibers,
separately to the formation of the pre-blanket, prepare an antiviral and/or antibacterial mixture in emulsion comprising the steps of:
optionally mixing about 0.1% to about 5% of at least one stabilizing additive, about 60% to about 95% of at least one polymer, and optionally at least one solvent in q.s.p (a sufficient quantity) 100% by weight, under stirring, forming a first polymeric emulsion,
adding about 0.1% to about 10% of at least one pigment and optionally about 1% to about 10% of at least one catalysis additive to the first polymeric emulsion under agitation,
adding at least one antiviral and/or antibacterial agent to the first polymeric emulsion under agitation, forming the antiviral and/or antibacterial mixture into an emulsion, the concentrations being relative to the total weight of the second polymeric emulsion.
after homogenizing the antiviral and/or antibacterial mixture in emulsion, keep stirring for at least about 10 minutes,
incorporate the antiviral and/or antibacterial emulsion mixture into the non-abrasive non-woven pre-blanket,
perform a first curing step on the pre-blanket incorporated with the antiviral and/or antibacterial mixture at least about 100° C. to about
250° C. at an air speed of about 5 m/s to about 10 m/s, forming an antiviral blanket and/or antibacterial;
(II) form an antiviral and/or antibacterial abrasive blanket comprising the steps of:
preparing an emulsion abrasive mixture comprising the steps of:
optionally mixing about 0.1% to about 5% of at least one stabilizing additive, about 10% to about 50% of at least one polymer, and optionally at least one solvent in q.s.p (a sufficient quantity) 100% by weight, under stirring, forming a second polymeric emulsion,
adding about 0.1% to about 10% of at least one pigment and optionally about 1% to about 10% of at least one catalysis additive to the second polymeric emulsion under agitation,
adding about 40% to about 80% of abrasive grains abrasive grains to the second polymeric emulsion under agitation, forming an abrasive mixture in emulsion, the concentrations being relative to the total weight of the second polymeric emulsion.
after homogenizing the abrasive mixture into an emulsion, keep stirring for at least about 10 minutes,
incorporate the abrasive mixture in emulsion into the non-abrasive non-woven antiviral and/or antibacterial blanket,
performing a second curing step on the antiviral and/or antibacterial blanket incorporated with the abrasive mixture at least about 100° C. to about 250° C. at an air speed of about 5 m/s to about 10 m/s,
(III) bonding the antiviral and/or antibacterial abrasive blanket to a foam that optionally comprises an antiviral and/or antibacterial agent.

24. Process according to claim 23, characterized in that the stirring is from about 1000 rpm to about 2000 rpm, and in which the post-homogenization stirring of the emulsion mixtures is maintained for about 10 to about 30 minutes, preferably about 20 minutes.

25. Process according to claim 23 or 24, characterized in that the first and second stages of curing are carried out at 180° C. at an air velocity of about 6 m/s to about 8 m/s.

26. Process according to any one of claims 23 to 25, characterized in that the fibers of the non-woven pre-blanket are synthetic fibers selected from polyamide, polyester, or similar fibers or combinations thereof.

27. Process according to any one of claims 23 to 26, characterized in that the first and second polymeric emulsions additionally comprise at least one stabilizing additive and/or at least one catalysis additive.

28. Process according to any one of claims 23 to 27, characterized in that the at least one polymer is selected from acrylic, phenolic, vinyl, polyolefins, polyurethanes, or their copolymers or similar or mixtures thereof.

29. Process according to any one of claims 23 to 28, characterized in that the at least one stabilizing additive is selected from thickeners, anti-foaming agents, preservatives, salts, co-surfactants, or similar and combinations thereof, preferably being selected thickeners and defoamers.

30. Process according to any one of claims 23 to 29, characterized in that the at least one catalysis additive is selected from alkylated urea, alkylated melamine-formaldehyde, alkylated urea-formaldehyde, benzoguanamine-formaldehyde, glycol-uryl-formaldehyde, or similar and their combinations, preferably being methylated melamine-formaldehyde.

31. Process according to any one of claims 23 to 30, characterized in that the at least one antiviral and/or antimicrobial agent is selected from nanoparticles with a nucleus composed of metallic atoms and/or organic molecules comprising ammonium quaternaries, cationic polysaccharides, saturated dialdehydes, isothiazolinones, pure or in mixtures, their similar and combinations thereof,
wherein at least one dimension of the nanoparticles having a core composed of metal atoms is from about 1 nm to about 100 nm and the metal atoms are selected from copper, silver, gold, zinc or the like or combinations thereof, and
in which the nanoparticles can be stabilized with organic molecules selected from monomers, polymers, polyphenols, polyflavonoids, organic acids, silanes, siloxanes, or similar and combinations thereof.

32. Process according to any one of claims 23 to 31, characterized in that the foam is a polyurethane or cellulose-based foam, preferably a polyurethane foam.

33. Process according to any one of claims 23 to 32, characterized in that the bonding of the abrasive blanket and the foam is made with a solvent-free adhesive, preferably solvent-free polyurethane adhesive.

34. Use of an antiviral and/or antibacterial abrasive blanket, characterized by the fact that it is for cleaning soft or hard surfaces, and
wherein the antiviral and/or antibacterial abrasive blanket comprises a non-abrasive non-woven formed from synthetic and/or natural fibers and at least two polymeric emulsions,
wherein the non-abrasive non-woven is soaked in a first polymeric emulsion comprising an antiviral and/or antibacterial blend
comprising:
about 60% to about 95% of at least one polymer,
about 0.1% to about 5% of at least one stabilizing additive,
about 0.1% to about 10% of at least one pigment,
about 1% to about 10% of at least one catalyst additive,
about 0.1% to about 10% of at least one antiviral and/or antibacterial agent, and
optionally at least one solvent in q.s.p (a sufficient
quantity) 100% by weight,
the concentrations being relative to the total weight of the first polymeric emulsion,
and then soaked in a second polymeric emulsion comprising an abrasive mixture comprising:
about 10% to about 50% of at least one polymer,
about 0.1% to about 5% of at least one stabilizing
additive,
about 0.1% to about 10% of at least one pigment,
about 1% to about 10% of at least one catalyst additive,
about 40% to about 80% abrasive grains, and optionally at least one solvent in q.s.p (a sufficient quantity) 100% by weight,
the concentrations being relative to the total weight of the second polymeric emulsion.

35. Use according to claim 34, characterized by the fact that the soft or hard surfaces are floors or coatings, tiles, leather, clothing, fabrics or non-woven, crockery and household items.

36. Use of an antiviral and/or antibacterial cleaning sponge, characterized by the fact that it is for cleaning soft or hard surfaces, and
wherein the antiviral and/or antibacterial cleansing sponge comprises a first side and a second side,
wherein the first side comprises a foam optionally comprising an antiviral and/or antibacterial agent and the second side comprises a non-woven antiviral and/or antibacterial abrasive blanket,
wherein the non-woven antiviral and/or antibacterial abrasive blanket comprises natural and/or synthetic fibers and is soaked in at least two polymeric emulsions,
wherein the non-woven is first soaked in a first polymeric emulsion comprising an antiviral and/or antibacterial blend comprising:
about 60% to about 95% of at least one polymer,
about 0.1% to about 5% of at least one stabilizing additive,
about 0.1% to about 10% of at least one pigment,
about 1% to about 10% of at least one catalyst additive,
about 0.1% to about 10% of at least one antiviral
and/or antibacterial agent, and
optionally at least one solvent in q.s.p (a sufficient
quantity) 100% by weight,
the concentrations being relative to the total weight of the
first polymeric emulsion,
and then soaked in a second polymeric emulsion comprising an abrasive mixture comprising:
about 10% to about 50% of at least one polymer,
about 0.1% to about 5% of at least one stabilizing
additive,
about 0.1% to about 10% of at least one pigment,
about 1% to about 10% of at least one catalyst additive,
about 40% to about 80% abrasive grains, and optionally at least one solvent in q.s.p (a sufficient quantity) 100% by weight,
the concentrations being relative to the total weight of the second polymeric emulsion.

37. Use according to claim 36, characterized by the fact that the soft or hard surfaces are floors or coatings, tiles, leather, clothing, fabrics or non-woven, crockery and household items.

38. Antibacterial and/or antiviral abrasive blanket, characterized in that it is produced by the process as defined in any one of claims 14 to 22.

39. Antiviral and/or antibacterial cleaning sponge characterized by the fact that it is produced by the process as defined in any one of claims 23 to 33.
